(19) United States
(12) Patent Application Publication
(10) Pub. No.: US 2026/0315027 A1
(43) Pub. Date: Oct. 15, 2026

(54) LUNG TOMOGRAPHY DEVICE AND SYSTEM AND ASSOCIATED TOMOGRAPHY METHOD

(71) Applicants: INSTITUT POLYTECHNIQUE DE GRENOBLE, Grenoble (FR); UNIVERSITE GRENOBLE ALPES, Saint Martin D'Heres (FR); CENTRE NATIONAL DE LA RECHERCHE SCIENTIFIQUE, Paris (FR)

(72) Inventors: Cornel IOANA, Grenoble (FR); Angela DIGULESCU POPESCU, Grenoble (FR)

(21) Appl. No.: 19/484,509

(22) PCT Filed: May 14, 2024
(86) PCT No.: PCT/EP2024/063216

§ 371 Date: Nov. 14, 2025

(30) Foreign Application Priority Data
May 17, 2023 (FR) 2304938

(51) Int. Cl. A61B 8/08; A61B 8/00; A61B 8/13; G01S 7/52; G01S 15/89

(52) U.S. Cl. CPC A61B 8/08; A61B 8/13; A61B 8/4209; A61B 8/4477; A61B 8/58; G01S 7/5205; G01S 15/8956; G01S 15/8993

(57) ABSTRACT

The invention relates to a device and a system for tomography of the lungs, the device comprising a support and at least five transceivers fastened onto the support configured to transmit and receive an ultrasonic wave having a frequency variable over a frequency range, the transceivers being arranged according to at least two first assemblies each positioned facing a lung of a human or animal body. The associated tomography method comprises the transmission and the reception of the ultrasonic wave according to transmission and reception profiles, by the transceivers, and an analysis of the deformation between these profiles to obtain the speed of the wave according to the frequency and the direction of propagation of the wave, and construct a tomography on the basis of a piece of data relative to this speed.

### TECHNICAL FIELD OF THE INVENTION

The present invention relates to a device for tomography of the lungs, a tomography system comprising it, and a method for tomography of the lungs. More particularly, it is used for lung tomography in the medical field, for example for monitoring lung diseases whether in the hospital or at a doctor's office.

### PRIOR ART

The imaging of human organs is a very common medical procedure allowing diagnosis but also the study of the progression of diseases. In lung examinations, X-ray imaging is the imaging means used the most.

X-rays are generated by specific tubes at very high frequencies (typically between 3 EHz and 30 EHz with 1 EHz=1018 Hz) and very low wavelengths (typically between 1 nm and 10 pm), which explains both their penetration capacity and the very high resolution that allows to visualise anomalies having a very small size (approximately one μm). The principle of X-ray imaging is based on the absorption of X-rays according to the density of the tissues, itself related to the concentration of electrons. The regions of the body with a higher electron density absorb more X-rays and, on an imaging film, this produces a lighter zone. Conversely, the regions with a low electron density absorb less X-rays and the corresponding zones appear darker in the image.

Conventional X-ray imaging has some limitations. A first limitation stems from the two-dimensional nature of the representation of naturally three-dimensional structures. This does not allow to precisely measure the size and the position of the regions of interest for diagnosis. A second limitation stems from the impossibility of quantifying the densities of the regions through which the rays pass.

To overcome these limitations, computer-assisted tomography (commonly designated by the term Computer Tomography-abbreviated as CT) is currently widely used. This technique provides high-resolution, three-dimensional images allowing better diagnosis. Via the rotation of the emission system and the activation of several receivers, both controlled by the computer, it is possible to image the object through several planes and thus provide a 3D image.

Although it has very good performance for an accurate diagnosis of lung diseases, CT remains a method of investigation with ionising rays. This limits its use in terms of exposure frequency. In addition, it is a relatively costly method, because of the cost of the equipment and the cost of the medical procedure.

In order to reduce the exposure of patients to ionising rays, especially when the zone to be explored is large and examinations must be carried out frequently, imaging of the ultrasound type represents a promising solution.

With the COVID-19 pandemic, researchers found correlations between lung ultrasounds and the data obtained by CT. During a conventional ultrasound, an ultrasound transducer is applied onto the patient by a practitioner to obtain the image of a part of the lungs. Although less constraining and cheaper than CT, an ultrasound requires prolonged contact with patients with risks of contamination. Moreover, the number of procedures that can be carried out is limited, since an ultrasound requires the participation of a specialist practitioner, typically for 20 to 40 minutes.

The document US 2005/020918 A1 describes a device for imaging a patient comprising ultrasonic transducers in order to reconstruct a 3D image from detected echoes.

The document Demi, Libertario, et al. “Real-time multi-frequency ultrasound imaging for quantitative lung ultrasound-first clinical results.” The Journal of the Acoustical Society of America 148.2 (2020): 998-1006 describes an example of ultrasound imaging of lungs, based on a pulse-echo protocol and using a conventional ultrasound probe.

One goal of the invention is therefore to provide an autonomous lung imaging solution with better performance than the techniques based on ultrasounds, in particular to limit the need for the presence of the practitioner and/or offer the possibility of continuous monitoring, and in particular an improved autonomous lung imaging solution.

### SUMMARY OF THE INVENTION

To achieve this goal, according to a first aspect a device for lung tomography is provided, comprising
a support, and
at least five transceivers fastened onto the support and configured to transmit and receive an ultrasonic wave of variable frequency in a frequency range spanning from 1 MHz to 20 MHz, the transceivers being arranged according to at least two first assemblies each facing a lung of a human or animal body.

The device thus allows the transmission and the reception of ultrasonic waves between the various transceivers, for their propagation in the human or animal body at its lungs. Since the transceivers are fastened onto the support, they form a network having a determined geometry allowing the collection of information in the volume represented by the lungs via the propagation of the ultrasonic waves in this organ. Via this network of transceivers, the device can be placed on the human or animal body and then the measurements can be carried out autonomously without requiring the action of a practitioner. Since the measurements can be taken autonomously, continuous monitoring of the state of the lungs is made possible, for example in the case of hospitalisation. Relative to the conventional CT techniques, the device remains less expensive and easier to use, which makes this technique accessible in both facilities of the hospital type and smaller facilities such as an office in the city.

More particularly, said transceivers are configured to transmit an ultrasonic wave having a variable instantaneous frequency varying over time over a frequency range spanning from 1 MHz to 20 MHz, according to a determined profile called “transmission profile”, and receive a signal to form an instantaneous frequency profile varying over time over a frequency range spanning from 1 MHz to 20 MHz, called “reception profile”, the transceivers being arranged according to at least two first assemblies intended to each be positioned facing a lung of a human or animal body.

A second aspect relates to a lung tomography system comprising:
the device as introduced above,
treatment means capable of:
   sending, to a transceiver, an instruction to transmit an ultrasonic wave having a transmitted frequency varying over a frequency range spanning from 1 MHz to 20 MHz, according to a determined profile of frequency variation over time, called “transmission profile”,
   receiving, from at least one other transceiver, a signal to form a profile of frequency variation over time, called “reception profile”, of the ultrasonic wave as received by the at least one other transceiver,
   analyzing the deformation between the transmission profile and the reception profile, preferably between the transmission profile and each reception profile, so as to determine the speed of propagation of the wave according to the frequency and according to at least one propagation direction corresponding to the relative placement of the at least one other transceiver, preferably each other transceiver, relative to the transceiver emitting the ultrasonic wave,
   constructing a lung tomography according to a piece of data relative to the speed of propagation of the wave according to the frequency and the direction of propagation.

The system presents the same effects and advantages as the device according to the first aspect and, like the device, allows the implementation of the method according to the following aspect.

A third aspect relates to a method for tomography of the lungs of a human or animal body, comprising:
an application of a device according to the first aspect onto the torso of the human or animal body, so as to place each of the two first assemblies facing one of the two lungs of the human or animal body,
a plurality of measurements, each measurement comprising:
   a transmission, by a transceiver, of an ultrasonic wave having a transmitted frequency varying over a frequency range spanning from 1 MHz to 20 MHz, according to a determined profile of frequency variation over time, called “transmission profile”,
   a reception, by at least one other transceiver, of the transmitted ultrasonic wave to form a profile of frequency variation over time of the transmitted ultrasonic wave, called “reception profile”,
      the transceiver transmitting the ultrasonic wave being distinct between at least two successive measurements,
for each measurement, an analysis of the deformation between the transmission profile and the reception profile, so as to determine a piece of data that is a function of the speed of propagation of the wave, for example the speed of propagation of the wave, according to the frequency and in at least one direction of propagation corresponding to the relative placement of the other transceiver relative to the transceiver transmitting the ultrasonic wave,
construction of a lung tomography according to a piece of data relative to the speed of the wave according to the frequency and the direction of propagation.

The plurality of measurements, with sequential changes of the transceiver transmitting the ultrasonic wave between at least two successive measurements, allows to have distinct points of origin for the various ultrasonic waves transmitted, and thus several directions of transmission-reception. The propagation of each ultrasonic wave in the human or animal body allows to obtain a reception profile that depends on the local state of the patient's lung along these pathways of propagation of the wave. In particular, the speed of propagation of the wave changes according to the frequency according to the state of the lung, in the zone through which the ultrasonic wave passes. The method allows, on the basis of the reception profile and the transmission profile, to obtain the speed of propagation of the wave in a considered zone of the lung as a function of the frequency of the wave, according to the relative placement of the transceiver transmitting the wave and the transceiver(s) receiving the wave. A tomography of the lung can thus be reconstructed.

Since the device comprises a network of fastened transceivers, the method allows a lung tomography without having to manually move a probe or the transceivers. The method can be carried out autonomously without requiring the action of a practitioner during the measurements. Since the measurements can be taken autonomously, continuous monitoring of the state of the lungs is made possible, for example in the case of hospitalisation. Like for the previous aspects, with respect to the conventional CT techniques, the method remains easier to use and with a reduced cost, which makes this technique accessible in both facilities of the hospital type and smaller facilities such as an office in the city. The method can implement the tomography system according to the preceding aspect.

A fourth aspect relates to a computer program product comprising instructions that, when executed by at least one processor, carry out at least the steps of analyzing the deformation between the transmission profile and the reception profile of the measurements, and constructing the lung tomography of the method according to the third aspect.

According to one example, the instructions further execute the step of analyzing the deformation between the calibration transmission profile and the calibration reception profile. A fifth aspect relates to a method for diagnosing and/or monitoring a pulmonary state, for example a lung disease, comprising the method according to the third aspect.

According to one example, the diagnosis and/or monitoring method comprises, after the construction of the tomography, a diagnosis of a pulmonary state, for example an identification of an affected zone of the lung.

According to one example, the diagnosis and/or monitoring method comprises the construction of several tomographies at distinct times. The method can comprise the comparison of tomographies carried out at distinct times to monitor the change of the pulmonary state, preferably in an affected zone of the lung.

### BRIEF DESCRIPTION OF THE DRAWINGS

The goals, objects, as well as the features and advantages of the invention will be clearer from the detailed description of one embodiment thereof which is illustrated by the following appended drawings, in which:

FIG. 1 shows a schematic diagram of ultrasound imaging.

FIG. 2 shows a diagram of the device positioned on the patient, during a measurement of the tomography method, according to an exemplary embodiment.

FIG. 3 shows a diagram of the device positioned on the patient, during a measurement of the tomography method other than that illustrated in FIG. 2, according to an exemplary embodiment.

FIG. 4 shows a diagram of the device positioned on the patient, during another measurement of the tomography method, according to another exemplary embodiment in which each transceiver, other than that transmitting the ultrasonic wave, receives a reception profile.

FIG. 5 shows a schematic diagram of the propagation of the ultrasonic wave in the tomography method according to an exemplary embodiment.

FIGS. 6A and 6B illustrate an example of frequency distribution over time for an affected lung zone and a healthy lung, respectively.

FIG. 7 is a graph of the speed of propagation of the ultrasonic wave according to the frequency, for various pulmonary states and according to an exemplary embodiment.

FIG. 8 illustrates an example of tomography established on the basis of the speed of propagation of the ultrasonic wave.

FIG. 9 is a diagram describing steps of the tomography method, according to an exemplary embodiment.

FIGS. 10A and 10B show the device for lung tomography, according to two exemplary embodiments.

The drawings are given as examples and do not limit the invention. They constitute schematic diagrams intended to facilitate the understanding of the invention and are not necessarily plotted to the scale of the practical applications. In particular, the relative dimensions of the transceivers and of the lung tomography device are not necessarily representative of reality.

### DETAILED DESCRIPTION OF THE INVENTION

Before starting a detailed review of embodiments of the invention, optional features that can optionally be used in combination or alternatively are listed below.

According to one example, the transceivers are arranged so that the distance d between the nearest neighbouring transceivers is less than or equal to 10 cm.

According to one example, the transceivers are arranged according to the at least two first assemblies, and in addition a second assembly comprising at least one transceiver and disposed between said first assemblies. The two first assemblies are thus each facing a lung, and the transceiver(s) of the second assembly can be disposed facing other organs of the respiratory system, for example the larynx, the trachea, and/or one or more bronchi. The data acquired is thus more complete.

According to one example, the number of said transceivers is greater than or equal to seven. According to one example, the number of said transceivers is less than or equal to twelve, preferably less than or equal to nine. The number of said transceivers can be between seven and twelve, and more preferably between seven and nine. During the development of the invention, it was brought to light that these numbers of transceivers were sufficient to construct a sufficiently detailed tomography of the lungs. The more the number of transceivers is increased, the more detailed the tomography obtained, at least until the physical limit of installation of the network of transceivers is reached. In order to limit the complexity and the cost of the device, it was brought to light that for the construction of a lung tomography, a number of transceivers less than or equal to twelve, and preferably less than or equal to nine, was sufficient. Indeed, the device requires few transceivers, since they are configured to operate in pairs between a transmitter and one or more receivers and thus be able to measure the spectral dispersion of frequency profiles between one transmission point and another reception point. The device thus differs from the existing echo-detection imaging solutions, in which the number of receivers is significantly higher to be able to capture signals and create the image. According to one example, the transceivers of the two first assemblies are arranged symmetrically relative to a median axis between the two first assemblies. A symmetrical network of transceivers facing each lung is thus obtained. This thus facilitates the construction of a tomography of the lungs since this symmetrical arrangement is linked to the anatomy of the human respiratory system, which is substantially symmetrical.

According to one example, the support forms a piece of clothing, for example a vest or an apron. The device can thus be put on by the patient themselves, or by a person helping them, simplifying the placement and the keeping in place of the device on the patient and thus the autonomous execution of the tomography method.

According to one example, the support comprises a sheet or a piece of fabric. The support can thus easily conform to the human or animal body.

According to one example, the support comprises means for external attachment to the human or animal body. The device can thus be kept in place despite possible movements of the body, which facilitates the measurements and in particular the acquisition of measurements distributed over time, for example during continuous monitoring.

According to one example, the device comprises a controller configured to vary, preferably in a continuous manner, the instantaneous transmission frequency of the transceivers to form the transmission profile.

According to one example, the transmission profile is continuous in terms of frequency.

According to one example, the instantaneous frequency of the transmission profile is varied, preferably continuously, over a frequency range extending over at least 4 MHz, in the range between 1 MHz and 20 MHz. Equivalently, the difference between a highest frequency value and a lowest frequency value of the transmission profile is greater than or equal to 4 MHz. According to one example, a change in the speed of propagation of the wave is determined according to the frequency and in at least one direction of propagation corresponding to the relative placement of the at least one other transceiver, preferably each other transceiver, relative to the transceiver transmitting the ultrasonic wave. The tomography constructed can be a function of each determined change in speed.

According to one example, during the transmission of the ultrasonic wave, during the plurality of measurements and/or during the calibration step, the transmission profile comprises a non-linear variation of the frequency over time. This allows to limit the influence of movements of the patient on the measurements.

According to one example, during the transmission of the ultrasonic wave, during the plurality of measurements and/or during the calibration step, the transmission profile comprises a variation in the frequency over time according to a cubic frequency modulation law. Cubic frequency modulation is indeed less sensitive to the Doppler effect. Cubic frequency modulation. The movements of the patient thus have even less impact on the measurements and/or the calibration with cubic frequency modulation.

According to one example, the method comprises, before the plurality of measurements, a calibration of the device placed on the human or animal body, comprising:
a plurality of acquisitions, each acquisition comprising:
   a transmission, by a transceiver, of an ultrasonic wave having a frequency varying over a frequency range spanning from 1 MHz to 20 MHz, according to a determined profile of frequency variation over time, called “calibration transmission profile”,
   a reception, by at least two other transceivers, of the transmitted ultrasonic wave to form a profile of frequency variation over time of the transmitted ultrasonic wave, called “calibration reception profile”,
for each acquisition, an analysis of the deformation between the calibration transmission profile and the calibration reception profile, so as to determine an optimal transmission profile, and more particularly increasing, preferably maximising, the amplitude of the reception profiles.

The calibration allows to improve the resolution of the tomography, in particular by determining for each receiver the frequency range and/or the relative phase shift with respect to the other receiver(s) that maximises the level of measured signals.

According to one example, the optimal transmission profile:
corresponds to a frequency modulation profile for which a maximisation of the deformation between the calibration transmission profile and the calibration reception profile is observed, and/or
corresponds to a transmission profile, the phase shift of which maximises the received signal, and/or
has a range of frequency variation over which a maximisation of the deformation between the calibration transmission profile and the calibration reception profile is observed.

According to one example, between the various measurements of the plurality of measurements, each transceiver transmits the ultrasonic wave at least once. Therefore, this multiplies the points of origin of the ultrasonic wave, and thus allows to carry out the tomography in a more precise and more complete manner.

According to one example, the reception of the reception profile is carried out by at least two, and preferably each, of the other transceivers, the speed of propagation of the wave according to the frequency being determined for propagation directions corresponding to the relative placements of the at least two, and preferably of each, of the other transceivers in relation to the transceiver transmitting the ultrasonic wave. The reception profile is thus obtained for several and preferably for each of the transceivers. This multiplies the points of reception of the ultrasonic wave and thus the measured paths of propagation of the ultrasonic wave in the lungs, to improve the information represented in the tomography.

According to a preferred example, the transmitted frequency is varied over a range between 1 MHz and 10 MHz. This range represents a better compromise between the propagation performance of the wave and the resolution that can be obtained. According to another example, the transmitted frequency is varied over a range between 10 MHz and 20 MHz. The transceivers can thus have smaller dimensions.

According to one example, each measurement of the plurality of measurements comprises an electronic beamforming, and in particular for at least a part and preferably for each reception profile. The electronic beamforming improves the spatial sensitivity and allows to obtain a tomography having increased spatial resolution.

According to one example, the calibration comprises an electronic beamforming, and in particular for at least a part and preferably for each calibration reception profile. The electronic beamforming thus allows during the calibration to optimise the phase shift to maximise the received signal. The resolution of the tomography is thus improved.

In the rest of the description, the term “on” does not necessarily mean “directly on”. Thus, when it is indicated that a part or that an element A bears “on” a part or an element B, this does not mean that the parts or elements A and B are necessarily in direct contact with one another. These parts or elements A and B can either be in direct contact or bear on one another via one or more other part(s). The same applies for other expressions for example such as the expression “A acts on B” which can mean “A acts directly on B” or “A acts on B via one or more other part(s)”.

In the present patent application, when it is indicated that two parts are distinct, this means that these parts are separate. They can be:
positioned at a distance from one another, and/or
mobile relative to one another and/or
rigidly connected to one another while being fastened by added elements, this fastening being disassemblable or not.

A unitary one-piece part cannot therefore consist of two distinct parts.

In the present patent application, the term “rigidly connected” used to qualify the link between two parts means that the two parts are linked/fastened with respect to one another, according to all degrees of freedom, unless otherwise explicitly specified. For example, if it is indicated that two parts are rigidly connected in translation according to a direction, this means that the parts can be mobile relative to one another, possibly according to several degrees of freedom, excluding the freedom in translation according to the direction. In other words, if a part is moved in the direction, the other part carries out the same movement. Several embodiments of the invention implementing successive steps of the manufacturing method are described below. Unless explicitly mentioned, the adjective “successive” does not necessarily mean, although this is generally preferred, that the steps immediately follow each other, and they can be separated by intermediate steps.

Moreover, the term “step” means the carrying out of a part of the method, and can designate a group of substeps.

Moreover, the term “step” does not necessarily mean that the actions carried out during a step are simultaneous or immediately successive. Certain actions of a first step can in particular be followed by actions related to a different step, and other actions of the first step can be taken up again afterwards. Thus, the term step does not necessarily mean unitary actions that are inseparable over time and in the sequence of the phases of the method.

A parameter “substantially equal to/greater than/less than” a given value means that this parameter is equal to/greater than/less than the given value, to within more or less 10% of this value. A parameter “substantially between” two given values means that this parameter is at least equal to the lowest value given, to within more or less 10% of this value, and at most equal to the highest value given, to within more or less 10% of this value.

The term “ultrasonic wave” is used for an acoustic wave, that is to say a mechanical vibration propagating in a medium, in a frequency range between 1 MHz and 20 MHz.

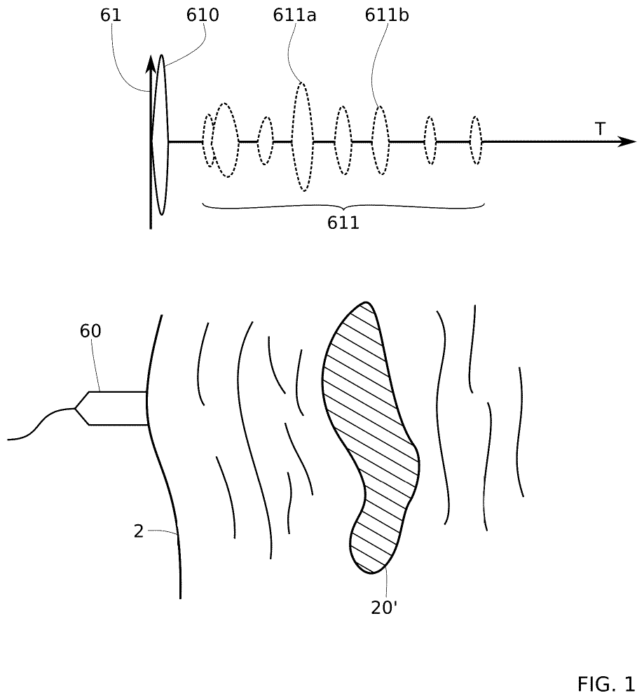

The general principle of imaging via ultrasonic waves, and more particularly via ultrasounds, is first described in reference to FIG. 1.

An ultrasound probe 60 is used, comprising several transceivers of ultrasonic waves, typically in the range of 1 MHz to 10 MHz generally used in ultrasound imaging. The ultrasound probe 60 is moved by a specialist practitioner over the skin of the body 2. During the measurement 61, the ultrasonic waves are transmitted 610 and echo 611a, 611b off the internal structures of the body 2, in particular off an organ to be imaged 20′. Several two-dimensional scans are carried out and they each constitute an ultrasound image. The basic principle is that the organ of interest 20′ and its constituent parts are generally characterised by echoes of greater amplitude than the reflections off other tissues that are not sought to be imaged.

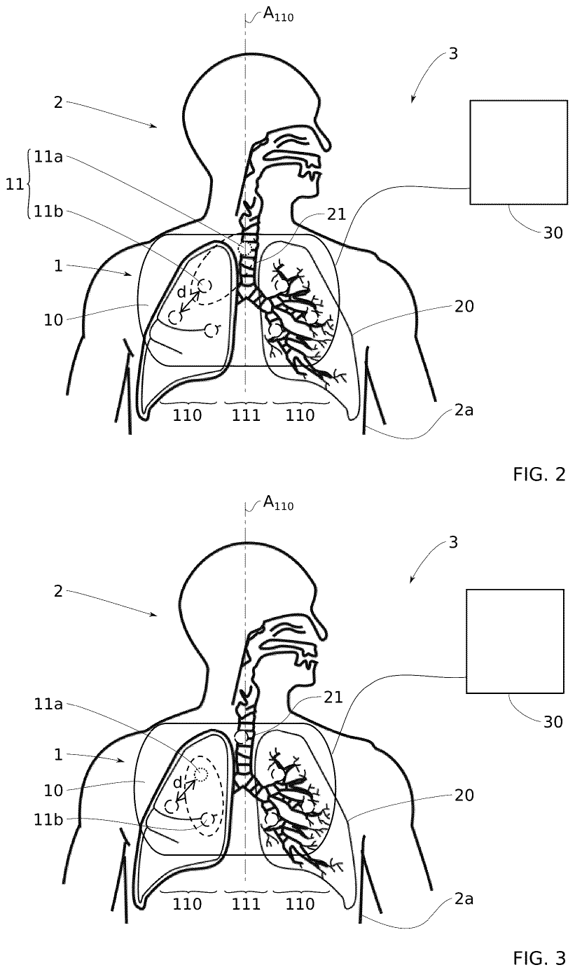

The device 1 for lung tomography according to the first aspect of the invention allows to eliminate the need for the presence and the manual operation carried out by the practitioner. The device 1 comprises a support 10 onto which transceivers 11, or equivalently “transducers”, are fastened in a removable manner or not, as illustrated by FIGS. 2 to 4. These transceivers 11 form a network of transceivers 11 with a determined geometry, rigidly connected to each other, at least partially superimposed over the lungs 20 of a human or animal body 2. For this, the transceivers 11 are divided into at least two first assemblies 110, intended to each be disposed facing a lung 20. Some transceivers 11 can further be arranged in a second assembly 111 disposed between the first assemblies 110. These transceivers 11 of the second assembly 111 can for example be disposed so as to be facing other structures 21 of the respiratory system, for example the larynx, the trachea, and/or one or more bronchi.

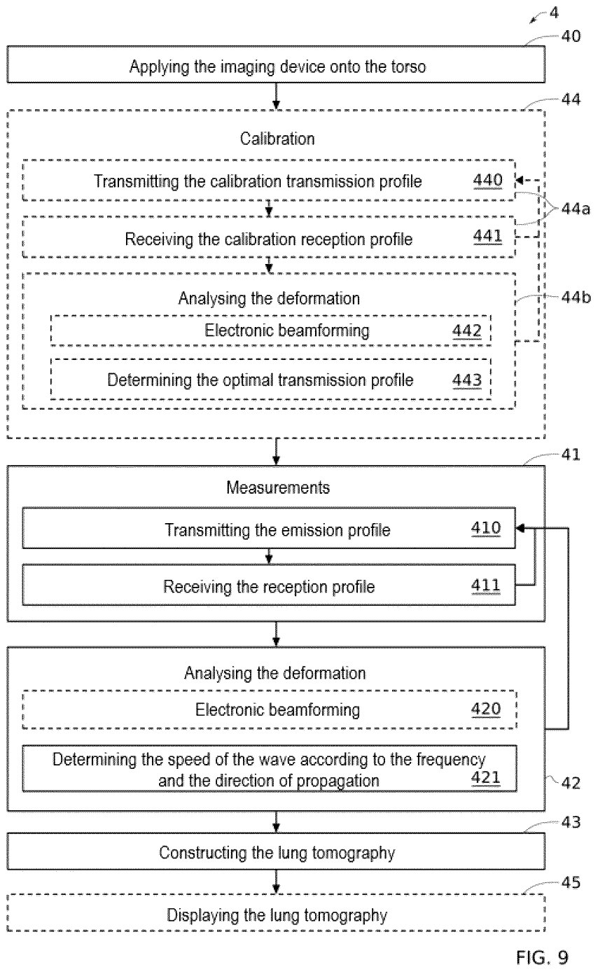

The operating principle of the invention is first of all described in general in reference to FIGS. 2 to 9, and specific embodiments are described. FIG. 9 describes steps of the method, in which optional steps are indicated by dotted lines and parallel paths and bifurcations indicate possible alternatives.

The device 1 is applied onto the torso 2a of a human or animal body 2, hereinafter designated as body 2. The device 1 can preferably be applied onto the chest, as illustrated in FIGS. 2 to 4. It is possible, although this is not preferred, for the device to be applied onto the back of the patient 2. The device 1 is placed so as to dispose each of the first assemblies 110 facing a lung 20.

After this placement, a transceiver 11a is configured to transmit an ultrasonic wave 12 having a frequency varying over a frequency range spanning from 1 MHz to 20 MHz, according to the time. The instantaneous frequency is therefore varied as a function of time, and preferably continuously, to form a determined profile. According to one example, the frequency profile is continuous, that is to say that it does not comprise any signal interruption. The instantaneous frequency varying over time is more particularly non-zero. The instantaneous frequency varying over time thus differs from frequency pulses isolated from each other. Preferably, the frequency is varied according to a coherent frequency sweep, that is to say according to a frequency path at least partially continuous and defined between an initial frequency and a final frequency. Equivalently, this is called modulation of the frequency of the ultrasonic wave 12. The transceiver 11a can transmit a wave 12 having a frequency varying over a frequency range narrower than the range between 1 MHz and 20 MHz. As explained in more detail later, the invention takes advantage of a modification of frequency profile between the transmission 410 of the wave 12 and its reception 411. For example, the wave varies over a range having a span greater than or equal to 4 MHz, for example of 6 MHz, for example of 5 MHz. This range is rather given by the processing capacity of the transceivers, for example between 1 MHz and 6 MHz, or between 5 MHz and 10 MHz. The instantaneous frequency can thus have a number of different values greater than or equal to 10, for example.

According to one example, the span comprising the range of frequency variation is between 1 MHz and 15 MHz, and preferably between 1 MHz and 10 MHz, corresponding to the band generally used in ultrasound imaging. In these ranges, the higher the frequency, the higher the resolution. However, the propagation performance can decrease for high frequencies. The frequency ranges used further depend on the transceiver technology used, in a manner known to a person skilled in the art.

To vary the frequency of the transmitted ultrasonic wave, the device 1 can comprise a controller configured to vary, preferably continuously, the instantaneous frequency of transmission of the ultrasonic wave 410 by the transceiver 11a to form the transmission profile. The controller can thus command the transceiver 11a to change the instantaneous frequency of the ultrasonic wave over time, according to the ranges indicated above for example.

As illustrated in FIG. 5, the ultrasonic wave 410 transmitted by a transceiver 11a propagates in the body 2, and in particular in the volume of the lungs 20. According to the nature and the state of the tissue encountered in the zone, noted as Ω_{m,n}, through which the ultrasonic wave 12 passes, the properties of the ultrasonic wave are modified. The zone Ω_{m,n} is also designated as volume Ω_{m,n}, with m the transceiver 11a and n the transceiver 11b. The determination of the modification of these properties allows the estimation of the information with a view to constructing the tomography 430 of the lungs 20.

More particularly, during a measurement 41, the transceiver 11a transmits 410 the ultrasonic wave 12 having a frequency varying over the frequency range, according to a determined profile for frequency variation over time. The instantaneous frequency transmitted varies over time to form this profile, called “transmission profile” 120. At least one other transceiver 11b, distinct from the transceiver 11a that transmits the ultrasonic wave 12, detects the ultrasonic wave 12 after its propagation in the tissue. This transceiver 11b detects the signal of the wave to form a profile of frequency variation over time, called “reception profile” 121. The instantaneous frequency detected by the receiver 11b varies over time, which constitutes the reception profile 121.

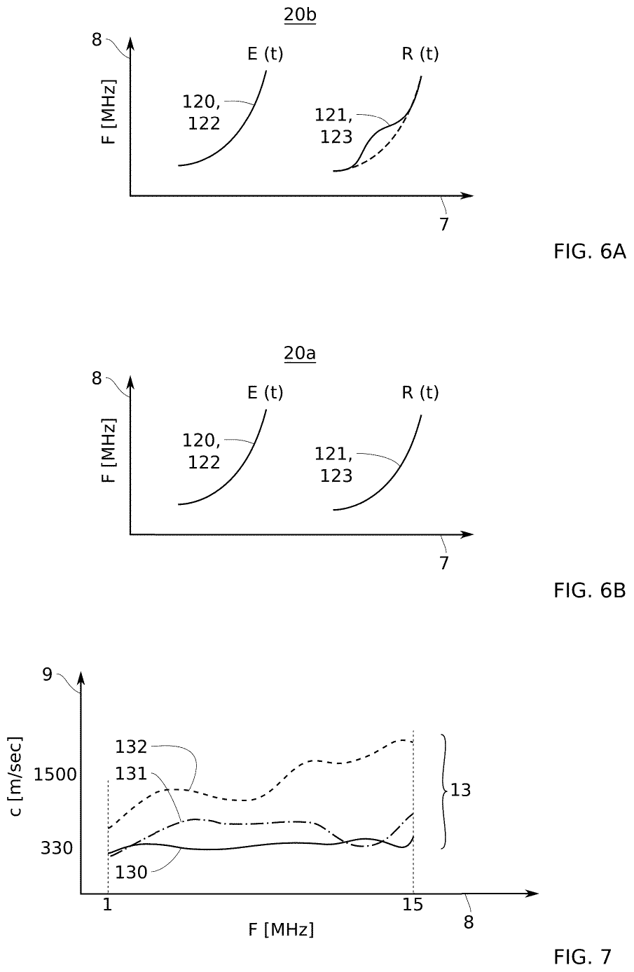

As an example, as illustrated by FIGS. 6A and 6B, the reception profile 121 can be deformed according to the nature of the tissue through which the ultrasonic wave 12 passes. Indeed, in a homogeneous medium, the speed of an ultrasonic wave is not dependent on the frequencies. In a heterogeneous medium, composed of several types of materials as is the case for the lungs 20, the speed of the ultrasonic wave 12 depends on the acoustic impedances of the various layers travelled through. These impedances influence the coefficients of transmission of the waves in the medium. The transmission speed thus varies, as well as the frequency of the wave, according to the heterogeneous medium passed through. It is therefore understood that a deformation of the reception profile 121 in relation to the transmission profile 120 is obtained according to the nature of the tissue passed through.

Typically, in the presence of air, the speed of propagation of an ultrasonic wave 12 is 330 m/sec while the speed of propagation of an ultrasonic wave 12 in water is 1480 m/sec. Pulmonary damage generally has the effect of congestion of the respiratory tract that obstructs the passage of air. An increase in the speed of propagation of the wave 12 will be observed, and an increase in the number of the propagated frequencies, since ultrasonic waves propagate more easily in tissue containing water.

In affected lung tissue 20b, the ultrasonic wave 12 passes through the potentially damaged tissue and a volume of air partially filled with secretions and phlegm. FIG. 6A illustrates as an example a deformation of the reception profile 121 with respect to the transmission profile 120, for affected lung tissue 20b. In healthy lung tissue 20a, the ultrasonic wave 12 passes through the tissue and a larger volume of air. FIG. 6B illustrates as an example a small deformation of the reception profile 121 relative to the transmission profile 120, for healthy lung tissue 20a. The reception profile 121 is less affected than in the case of affected lung tissue 20b.

Knowing the location of the transceiver 11a transmitting the ultrasonic wave 12, and that of the transceiver 11b receiving it, the analysis 42 of this deformation allows to obtain the speed of the wave 12 as a function of the frequency and according to a propagation direction corresponding to the relative placement of the transceiver 11b with respect to the transceiver 11a. The direction of propagation, or propagation volume noted as Ω_{m,n}, corresponds to the volume in which the ultrasonic wave propagates, with m the transceiver 11a and n the transceiver 11b. This volume can be at least partly included in a plane including two transducers, one in transmission and the other in reception.

Several measurements 41 are carried out, the transceiver 11a transmitting the ultrasonic wave being distinct between at least two successive measurements 41, as illustrated for example by FIGS. 2 and 3. The point of origin is therefore varied at least between two successive measurements. The speed of the wave 12 as a function of the frequency and of several propagation directions in the zone Ω_{m,n} is thus obtained by the analysis 42 for each measurement.

From the speed of the wave 12 as a function of the frequency and as a function of several propagation directions in Ω_{m,n}, an image of the zone passed through can be obtained. The image construction for the entire zone covered by the network of transducers can be continuous and allows to have, for a series of measurements 41, a representation of the state of the lungs of the patients. A tomography 430 can thus be obtained as shown by FIG. 8. This tomography represents the lung 20 according to a piece of data relative to the speed of the wave 12 as a function of the frequency and the direction of propagation. A person skilled in the art is capable of identifying and using any piece of data relative to the speed of the wave for the construction of the tomography 43. This piece of data can be a colour scale or a level of grey. In the example illustrated in FIG. 8 for example, the parts with congestion 430a are indicated by darker colours than unaffected parts 430b.

Steps of the method 4 are now described in more detail, according to exemplary embodiments.

During the plurality of measurements 41, the transceiver 11a transmitting the ultrasonic wave 12 is distinct between at least two successive measurements 41 of the plurality of measurements 41. When the plurality of measurements comprises more than two measurements 41, the same transceiver 11a can transmit 410 the ultrasonic wave 12 over several measurements 41, for example over two successive measurements 41.

At least one transceiver 11a of each first assembly 110 preferably transmits 410 the ultrasonic wave 12 during the plurality of measurements 41. Some transceivers of an assembly 110 can be too far from the other assembly 110 to detect the ultrasonic wave transmitted 410 well. Thus, at least one point of origin of the wave is used for each first assembly 110. At least one transceiver 11a of the second assembly 111 can transmit 410 the ultrasonic wave 12. Thus, a point of origin disposed between the two first assemblies 110 is used. This allows in particular to carry out the tomography of central zones of the lungs like the trachea or the larynx.

Preferably, each transceiver 11 transmits the ultrasonic wave 12 at least once during the plurality of measurements 41. The method thus uses the entirety of the network of transceivers 11 to multiply the points of origin for the propagation of the ultrasonic wave 12, and thus obtain information for an increased number of propagation directions.

Preferably, a single transceiver 11a transmits the ultrasonic wave 12 during a measurement in order to avoid interference between the reception profiles 121 and facilitate the analysis 42 of the deformation.

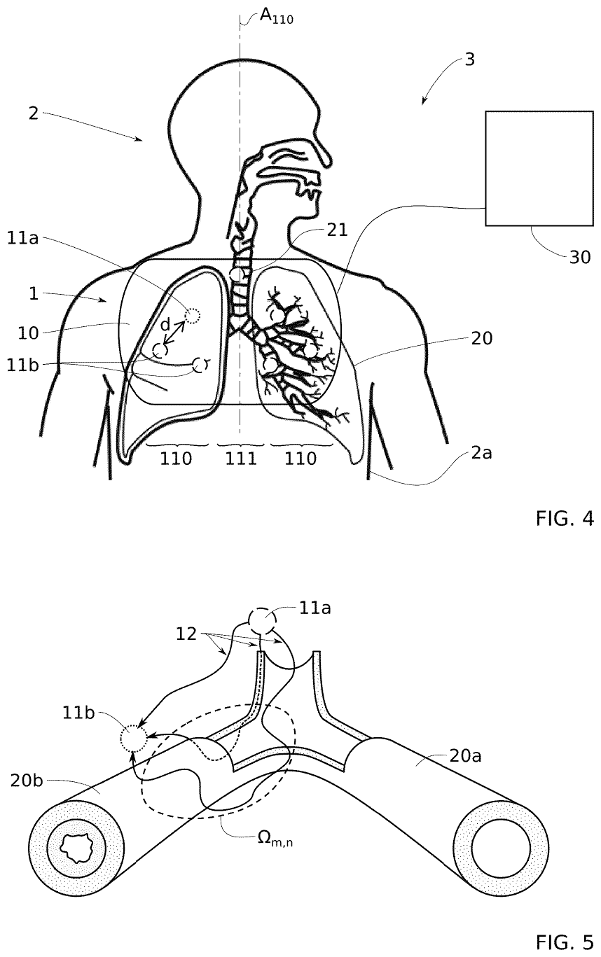

At least one transceiver 11b detects the ultrasonic wave 12 to obtain the reception profile 121. Preferably, several transceivers 11b detect the ultrasonic wave 12 to each obtain a reception profile 121. Thus, the points 411 of reception of the reception profiles 121 are multiplied. The same measurement 41 thus allows to obtain more information on the lung tissue passed through. More preferably, all the transceivers 11b, except for the one 11a that transmits the ultrasonic wave 12, detect the ultrasonic wave 12 to each obtain a reception profile 121, as illustrated in FIG. 4.

It is therefore understood that the network of transceivers 11 of the device 1 allows, via the set of various points of origin and of reception of the ultrasonic wave 12, to improve the spatial acquisition of the information in the pulmonary volume to be imaged, in a fast time. According to one example, there are at least as many measurements 41 as transceivers 11. For one measurement, for one device comprising seven transceivers 11, six transceivers 11b can receive the ultrasonic wave 12 for each measurement 41. Six reception profiles 121 can be obtained per measurement 41, and seven measurements can be carried out, or a total of 42 reception profiles for the seven measurements 41.

Synergistically between the number of measurements 41 with the number of distinct points of origin for the transmission 410 of the wave, and/or the number of reception points 411, both the number of reception profiles 121 and the number of associated propagation directions are increased. The spatial acquisition of the information in the pulmonary volume to be imaged is improved.

According to one example, the frequency modulation of the ultrasonic wave 12 follows a non-linear progression over time, for example as illustrated by FIGS. 6A and 6B. The transmission profile 120 can therefore be non-linear. This allows to limit the influence of the movements of the patient on the measurements. Non-linear modulation is indeed less sensitive to the Doppler effect since the polynomial coefficients associated with non-linear modulation are low. The transmitted 410 wave can be defined according to an amplitude and an instantaneous phase φ(t) characterising the non-linear modulation of the signal. More particularly, the frequency modulation of the ultrasonic wave 12 can follow a cubic progression.

For example, the transmitted wave E₍ₜ₎ can be defined by E₍ₜ₎=A·exp[j·φ₍ₜ₎], where A is the amplitude of the signal and φ(t) a function of time, j being the imaginary number such that j²=−1.

Upon reception, after propagation in the lung tissue and thus deformation of the modulation of the wave, the signal Rₖ(t) received by a receiver 11b can be a function of the amplitude A of the signal and of the instantaneous phase φ(t) over time, and also of a factor Lₖ/c^{m,n}ₖ(f), for a direction k.

The received signal R (t) can in particular have the expression:

$$R ( t ) \approx {\sum }_{k} {R}_{k} ( t ) = {\sum }_{k} {A}_{k} exp [ j \cdot Φ ( t - \frac{{L}_{k}}{{c}_{k}^{m , n} ( f )} ) ]$$

with:
Lk the propagation distances of the waves between a transceiver 11a, noted as m, transmitter of the wave, and a transceiver 11b, noted as n, receiver of the wave, and defined in the volume Ωm,n imaged by the transceiver pair 11a, 11b;
cm,nk represents the speed of the wave propagated in the direction k between the transmitter m and the receiver n, which depends on the frequency f and the propagation medium, these two elements being intrinsically related.

The analysis 42 of the deformation can be carried out between a reception profile 121 and a transmission profile 122, according to the conditions of which transceiver(s) 11a transmit 410 the ultrasonic wave 12 and which transceiver(s) receive it 411. Preferably, the analysis 42 of the deformation can be carried out between each reception profile 121 and the transmission profile 122.

This analysis 42 step can be at least partially simultaneous with the plurality of measurements 41, or separated temporally, as shown for example by the arrows looping back to the transmission 410 of the transmission profile in FIG. 9.

According to one example, the analysis of the deformation between a reception profile 121 and a transmission profile 122 can comprise an estimation of the modulation law of the signal of the reception profile 121. For this, a time-frequency-phase concept can be used in order to automatically and precisely extract the laws of non-linear modulations of a signal with several components. The details of this time-frequency-phase concept are described in detail in the document Cornel Ioana, et al. Localization in underwater dispersive channels using the time-frequency-phase continuity of signals. IEEE Transactions on Signal Processing, Institute of Electrical and Electronics Engineers, 2010, 58, pp. 4093-4107.

For example, a first step can be to adaptively build a local dictionary, by using polynomial phase modelling, for example cubic modelling. This allows to establish a correspondence between the instantaneous frequency laws of the polynomial modulations and the instantaneous frequency laws of the analyzed signal, for various analysis windows. A second step can then comprise merging the cubic modulations by using the maximisation of the local correlation between the signals extracted from the filters defined by the cubic modulations of the neighbouring analysis windows. This second step allows to automate the monitoring and minimise the propagation of errors.

More particularly, since the modulation law of the transmission profile 120 is known, the estimation of the modulation laws of the received signals Rₖ(t) above allows to estimate the deformations of the reception profile 122 in relation to the transmitted modulation 120.

The analysis 42 can comprise a determination of the variation of the speed c according to the frequency, which depends on the presence of the volume of the various materials passed through, in the volume Ω_{m,n} for a direction k, the direction k being according to the relative placement of the transceiver 11b receiving the wave 12 and the transceiver 11a transmitting it. This gives a frequency dispersion profile. For example, the estimation of the modulation laws of the signal of the reception profile 121 gives access for each frequency to the propagation speed in the directions k of the volume Ω_{m,n}. This dispersion profile indicates the variation of the speed c of each of the frequencies, which depends on the presence of the volume of the various materials passed through, in the volume Ω_{m,n} for the direction k.

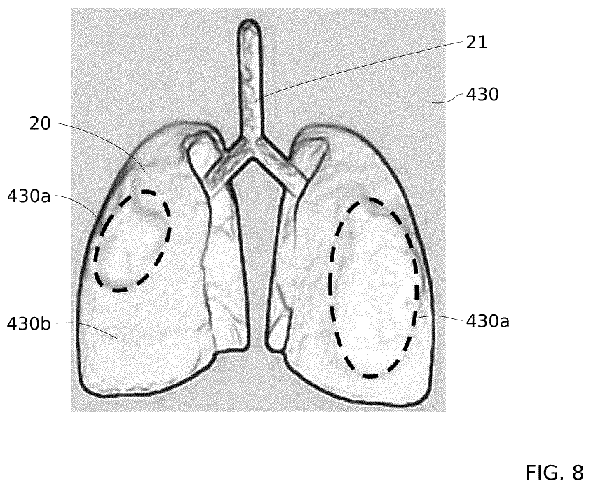

With this estimated dispersion profile, the analysis 42 can comprise a quantification of the amount of congestion in the volume Ω_{m,n} as illustrated for example by FIG. 7. In FIG. 7, the speed 9 according to the frequency 8 is represented for three cases:
A dispersion profile 130 for healthy lungs,
A dispersion profile 131 during the initial installation of the system on a patient, for lungs with an onset of pulmonary congestion,
A dispersion profile 132 at a time that corresponds to a monitoring interval, showing a change in the lung congestion.

It is therefore understood that a quantified piece of data on the pulmonary state can for example be obtained by analyzing the speed of propagation of the wave according to the frequency and the direction of propagation k.

An image 430 of the zone Ω_{m,n}, as described above, can be obtained by integrating the speed of the wave 12 as a function of the frequency over all the propagation directions k.

For example, this integration can be expressed analytically by

$${I}_{m , n} = {∮}_{\{ {L}_{k} \}} {\int }_{f 1}^{f 2} {c}_{k} ( f ) dfd \{ {L}_{k} \}$$

with {Lₖ} belonging to Ω_{m,n}, and f1 the lower limit and f2 the upper limit of the range of variation of the transmitted wave (for example, f1=1 MHz and f2=15 MHz).

Before the measurements 41, the method can further comprise a step of calibration 44 of the transmission profiles 120, which can equivalently be designated by the term “initialisation”. This calibration aims to determine the transmission profile 120 most suitable for acquiring information on the state of the lung tissue. For this, the calibration comprises a plurality of acquisitions 44a, each comprising:
a transmission 440, by a transceiver 11a, and in particular the one that will transmit the ultrasonic wave during the measurements 41, of an ultrasonic wave 12 having a frequency varying over the frequency range provided for the measurements 41, according to a frequency profile and over time called “calibration transmission profile” 122,
a reception 441, by at least two other transceivers 11b, of the transmitted ultrasonic wave 12, according to the frequency profile and as a function of the time of the transmitted ultrasonic wave 12, called “calibration reception profile” 123.

Indeed, an optimal reception configuration corresponds to that which allows to obtain a signal with maximal amplitude at the transceiver receiving the signal. To do this, it is for example possible to play on two parameters.

A first parameter is the band of the transmitted signal. The choice of a wide band (for example between 1 MHz and 15 MHz) allows to have a sufficiently broad range to guarantee that the maximum number of frequencies arrive at the transceiver receiving the signal with a sufficient level. Furthermore, the larger the frequency range, the more effective the measurement of dispersion.

A second parameter is the propagation direction. In the device, the transceivers are oriented towards the lungs and, between each one, the propagation medium is different from one patient to another. Since the transceivers are fixed, it is possible to play on the relative phase shifts between the receptions. This is the principle of electronic beamforming which allows to maximise the level of received signals by optimising the relative phase shifts between the transceivers. The phase shift can therefore be optimised so as to maximise the received signal level. During calibration, at least two other transceivers 11b can receive the ultrasonic wave 12 transmitted to analyze these relative phase shifts.

According to one example, the calibration reception profile 123 is received 441 by the transceivers 11b intended to receive 411 the reception profile 122 during the measurements 41, and preferably by all the transceivers 11b of the device 1 other than the one that transmits 440 the ultrasonic wave 12.

Between several acquisitions 44a, and preferably for the same transceiver 11a, the calibration transmission profile 122 can be distinct between at least two successive acquisitions 44a. An analysis 44b of the deformation between the calibration transmission profile 122 and the calibration reception profile 123 can be carried out. This deformation is for example shown by FIGS. 6A and 6B, similarly to the deformations described for the measurements step 41. Between several acquisitions 44a, and preferably for the same transceiver 11a, an optimised transmission profile can be determined.

This determination can be based on the calibration transmission profile 121 for which the amplitude of the received signals is maximal. The resolution of the tomography 430 is thus improved. In particular, it is possible to seek to maximise the deformation by the electronic beamforming and/or the range of frequency variation in the span described above.

The frequency modulation of the ultrasonic wave 12 transmitted 440 during the calibration can have the same features as those described in reference to the measurements 41.

This calibration 44 can be carried out for several and preferably for all the transceivers 11a intended to transmit 410 the ultrasonic wave 12 during the measurements 41. As described for the measurements 41, preferably a single transceiver 11a emits the ultrasonic wave during an acquisition 44a. For example, the calibration 44 is repeated for each of these transceivers 11a.

Here too, it is possible for this analysis step 44b to be at least partially simultaneous with the plurality of acquisitions 44a, or temporally separated, as shown as an example by the arrows looping back to the transmission 440 of the calibration transmission profile in FIG. 9.

During the measurements 41 and/or during the calibration 44, electronic beamforming 420, 442 (generally called adaptive beam forming) can be carried out on the reception profile(s) 121, 123.

As known in the field, the methods for electronic beamforming are techniques that allow to play on a relative phase shift. This is the principle of electronic beamforming which allows to maximise the level of received signals by optimising the relative phase shifts between the transceivers. This phase shift virtually allows to change the position of the transmitter of the ultrasonic. This therefore allows to obtain for a given position of a transmitter and of a receiver, several configurations between which the measured signal levels can vary. During the calibration, the phase shift can thus be optimised so as to identify the phase-shift configuration that maximises the received signal level. During the measurements, the electronic beamforming allows to introduce phase shifts, which artificially increases the propagation directions. The resolution of the resulting tomography is therefore increased.

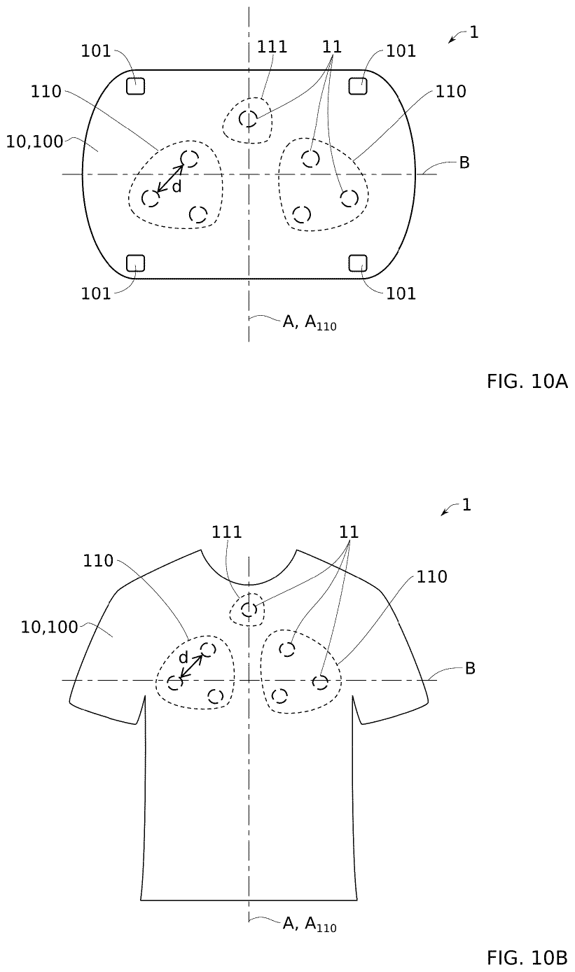

The device 1 is now described in more detail, according to several exemplary embodiments that can be illustrated by FIGS. 10A and 10B.

The device 1 comprises at least five transceivers 11, and preferably between five and twelve. For example, the device 1 comprises seven transceivers 11. In the network of transceivers thus formed, the distance d between the nearest neighbouring transceivers 11 can be chosen so as to distribute the transceivers above the lungs to be imaged. It is therefore understood that this distance d can be adapted according to the morphology of the human or animal body. The distance d can further be adapted according to the frequency of the ultrasonic wave. The distance d is at least greater than or equal to the wavelength of the ultrasonic wave. The distance d can be between several centimetres and several tens of centimetres, for example substantially greater than or equal to 5 cm and/or substantially less than or equal to 30 cm. For example, the distance d is substantially greater than or equal to 7 cm. The distance d can be substantially less than or equal to 20 cm, preferably 15 cm. For example, the distance d can be substantially equal to 10 cm. These distances along with the number of receivers allow good distribution of the transceivers 11 over the zone of the lungs 20 to be imaged. It should be noted that this distance d can vary in this range among the transceivers 11. For example, it is possible for the distance d to be reduced between certain transceivers, in order to increase the resolution of a given zone and obtain a more precise tomography.

The transceivers 11 are preferably all placed in the same plane parallel to or the same as the plane of the support 10. It should be noted that the plane of the support 10 can be curved to conform to the human or animal body. The distance between the transceivers 11 most distant from each other can be less than or equal to 50 cm, preferably 30 cm, in a lateral direction parallel to the direction B. This distance can be greater than or equal to 15 cm, preferably 20 cm. The distance between the transceivers 11 most distant from each other can be less than or equal to 50 cm, preferably 30 cm, in a longitudinal direction parallel to the direction A. This distance can be greater than or equal to 15 cm, preferably 20 cm. In each first assembly 110, the distance between the transceivers 11 most distant from each other can be less than or equal to 25 cm, preferably 15 cm. This distance can be greater than or equal to 5 cm, in a lateral direction parallel to the direction and/or a longitudinal direction parallel to the direction A. Here again, it is understood that these distances can be adapted according to the morphology of the human or animal body.

The two first assemblies 110 can be separated from one another, that is to say placed at a distance from one another in the plane of the support 10. They are for example separated by a distance greater than or equal to 5 cm, preferably 10 cm. The first assemblies 110 are preferably disposed symmetrically relative to a median axis A₁₁₀ between the two first assemblies 110. In this case, preferably the two first assemblies comprise an equal number of transceivers 11. The transceiver(s) 11 in the second assembly 11 can also be disposed symmetrically relative to this axis A₁₁₀. At least one transceiver 11 of this assembly 111 can be disposed on this axis for example.

According to one example, the support 10 forms a piece of clothing suitable for being put on by a patient, as illustrated in FIG. 10B. This piece of clothing can be an apron or a vest for example. In another example, the support 10 can be configured to simply rest on the torso 2a of the body 2, as illustrated by FIG. 10A.

The support 10 can comprise attachment means 101 external to the human or animal body 2, that is to say non-invasive. For example, these attachment means 101 can be straps, elastic belts or any other means that a person skilled in the art may consider.

The support 10 can be flexible, that is to say deformable manually without a tool, to conform to the body 2. The support 10 can be made from a sheet (for example of the paper or plastic type) or from a fabric 100. A person skilled in the art is capable of identifying and implementing a support capable of receiving the transceivers 11 in the device 1.

The tomography system 3, for example illustrated by FIGS. 2 to 4, comprises the device 1 and processing means 30 configured to send instructions to the transceivers 11 and receive the profiles received from the transceivers 11, in particular for the implementation of the steps of the method 4. It should be noted that it is possible for the reception means to receive the signal received by the transceivers 11b one piece of data at a time, and for the processing means 30 to form the reception profile 121, 123. These means 30 can further carry out the steps of analysis 42, 44b and constructing 43 the tomography described above. The system 3 can further comprise a display means (not shown in the drawings) on which the tomography 430 can be displayed 45. The processing means 30 can further comprise a memory in which the acquired and calculated data is stored, for example the reception profiles 121, 123 during the measurements 41 and/or acquisition 44a.

The communication between the device 1 and the processing 30 and/or display means can be carried out in a wired or wireless manner. The processing means 30 can be integrated into the device 1 or remote from the device 1.

These processing means 30 can for example comprise a computer program product comprising instructions allowing the implementation of the steps of the method 4, and in particular the steps of analyzing 42, 44b the deformation between the transmission profile 120, 122 and the reception profile 121, 123 during the measurements and/or the calibration 44, and of constructing 43 the tomography 430. The computer program product can further comprise instructions for coordinating the transmissions 410, 440 of an ultrasonic wave, and their reception 411, 441.

As directly clear from the above description, the computer program product can be a computer software stored on a non-transient medium readable by computer.

The invention is not limited to the embodiments described above and extends to all the embodiments covered by the invention. In particular, the device, the tomography system, and/or the computer program product can comprise any feature configured to allow the implementation of a step of the method, and conversely, the method can comprise any step resulting from the implementation of a feature of the device, of the tomography system and/or of the computer program product.

1. A device for tomography of lungs comprising:
a support, and
at least five transceivers fastened onto the support, and configured to transmit an ultrasonic wave having an instantaneous frequency varying over time in a frequency range spanning from 1 MHz to 20 MHz, according to a determined profile called “transmission profile”, and to receive a signal to form an instantaneous frequency profile varying over time over a frequency range spanning from 1 MHz to 20 MHz, called “reception profile”, the transceivers being arranged according to at least two first assemblies intended to each be positioned facing a lung of a human or animal body.

2. The device according to claim 1, wherein the transceivers are arranged according to the at least two first assemblies, and a second assembly comprising at least one transceiver and disposed between said first assemblies.

3. The device according to claim 1, wherein the number of said transceivers is between seven and twelve.

4. The device according to claim 1, wherein the transceivers of the two first assemblies are further arranged symmetrically relative to a median axis between the two first assemblies.

5. The device according to claim 1, wherein the support forms a piece of clothing.

6. The device according to claim 1, wherein the support comprises a sheet or a fabric and attachment means external to the human or animal body.

7. A system for tomography of the lungs comprising:
the device according to claim 1,
processing means configured to:
send, to a transceiver, an instruction to transmit an ultrasonic wave having a transmitted frequency varying over a frequency range spanning from 1 MHz to 20 MHz, according to a determined profile of frequency variation over time, called “transmission profile”,
receive, from at least one other transceiver, a signal, and to form a profile of frequency variation over time, called “reception profile”, of the ultrasonic wave as received by the at least one other transceiver,
analyze the deformation between the transmission profile and the reception profile, so as to determine the speed of propagation of the wave as a function of the frequency and according to at least one propagation direction corresponding to the relative placement of the at least one other transceiver with respect to the transceiver transmitting the ultrasonic wave,
construct a tomography of the lungs according to a piece of data relative to the speed of the wave according to the frequency and the direction of propagation.

8. A method for tomography of the lungs of a human or animal body, comprising:
an application of a device according to claim 1 onto the torso of the human or animal body, so as to place each of the two first assemblies facing one of the two lungs of the human or animal body,
a plurality of measurements, each measurement comprising:
a transmission, by a transceiver, of an ultrasonic wave having a transmitted frequency varying over a frequency range spanning from 1 MHz to 20 MHz, according to a determined profile of frequency variation over time, called “transmission profile”,
a reception, by at least one other transceiver, of the transmitted ultrasonic wave to form a profile of frequency variation over time of the transmitted ultrasonic wave, called “reception profile”,
the transceiver transmitting the ultrasonic wave being distinct between at least two successive measurements,
for each measurement, an analysis of the deformation between the transmission profile and the reception profile, so as to determine a piece of data that is a function of the speed of propagation of the wave according to the frequency and according to at least one propagation direction corresponding to the relative placement of the other transceiver in relation to the transceiver transmitting the ultrasonic wave,
a construction of a tomography of the lungs according to a piece of data relative to the speed of propagation of the wave as a function of the frequency and the propagation direction.

9. The method according to claim 8, wherein, during the transmission of the ultrasonic wave, the transmission profile comprises a non-linear variation of the frequency over time.

10. The method according to claim 9, wherein, during the transmission of the ultrasonic wave, the transmission profile comprises a variation of the frequency over time according to a cubic frequency modulation law.

11. The method according to claim 8, comprising, prior to the plurality of measurements, a calibration of the device placed on the human or animal body, comprising:
a plurality of acquisitions, each acquisition comprising:
a transmission, by a transceiver, of an ultrasonic wave having a frequency varying over a frequency range spanning from 1 MHz to 20 MHz, according to a determined profile of frequency variation over time, called “calibration transmission profile”,
a reception, by at least two other transceivers, of the transmitted ultrasonic wave, to form a profile of frequency variation over time of the transmitted ultrasonic wave, called “calibration reception profile”,
for each acquisition, an analysis of the deformation between the calibration transmission profile and the calibration reception profile, so as to determine a transmission profile that increases the amplitude of the reception profiles.

12. The method according to claim 8, wherein, between the various measurements of the plurality of measurements, each transceiver transmits the ultrasonic wave at least once.

13. The method according to claim 8, wherein the reception of the reception profile is carried out by at least two, and preferably each, of the other transceivers, the speed of propagation of the wave as a function of the frequency being determined for propagation directions corresponding to the relative placements of the at least two, and preferably each, of the other transceivers in relation to the transceiver transmitting the ultrasonic wave.

14. The method according to claim 8, wherein the transmitted frequency is varied over a range between 1 MHz to 10 MHz.

15. The method according to claim 8, wherein each measurement of the plurality of measurements comprises an electronic beamforming and/or the calibration comprises an electronic beamforming.

16. A computer program product comprising instructions, which when carried out by at least one processor, execute at least the steps of analyzing the deformation between the transmission profile and the reception profile of the measurements and constructing the tomography of the lungs of the method according to claim 8.

17. The computer program product according to claim 16, wherein the instructions further execute the step of analyzing the deformation between the calibration transmission profile and the calibration reception profile.

18. A method for diagnosing and/or monitoring a pulmonary state comprising the method according to claim 8.
